(19) United States
(12) Patent Application Publication
(10) Pub. No.: US 2024/0010868 A1
(43) Pub. Date: Jan. 11, 2024

(54) POLYMER ABLE TO FORM A SLIPPERY COATING FOR AN OPHTHALMIC INJECTOR

(71) Applicant: POLYMEREXPERT SA, Pessac (FR)

(72) Inventor: Marc DOLATKHANI, Cestas (FR)

(21) Appl. No.: 18/254,905

(22) PCT Filed: Nov. 30, 2021
(86) PCT No.: PCT/FR2021/052148

§ 371 Date: May 30, 2023

(30) Foreign Application Priority Data
Nov. 30, 2020 (FR) FR2012422

(51) Int. Cl. C09D 175/08; C08G 18/22; C08G 18/40; C08G 18/32; C08G 18/42

(52) U.S. Cl. CPC C09D 175/08; C08G 18/227; C08G 18/4018; C08G 18/3203; C08G 18/4277; A61F 9/00736

(57) ABSTRACT

The invention relates to a polymer, the molar masses and the hydrophilic/hydrophobic balance of which are adjusted, able to form a slippery coating on the internal surface of at least part of a device for injecting an intraocular implant. The invention also relates to the use of the polymer for coating the internal surface of one or more parts of a device for injecting an intraocular implant, the method for coating said surface, as well as a device for injecting an intraocular implant comprising at least one portion, the internal surface of which is coated with the polymer.

### TECHNICAL FIELD

The invention relates to the development of a slippery coating intended to coat at least one portion of an intraocular implant injection device, in particular the tip and/or the cartridge, enabling injection by micro-incision. The main property of this coating is to enable the folded implant to slip along the tip and/or the cartridge without peeling off and without being entrained into the eye.

### PRIOR ART

The replacement of the lens of the eye affected by cataract is ensured by intraocular implants. Surgery using phacoemulsification, enables the destruction of the natural lens and the elimination thereof through a small incision. Implants have been developed in flexible and foldable materials that could be inserted using injection devices through the micro-incision made for phacoemulsification. The injection system is composed of a tubular body in which the injection piston slides surmounted by a conical tip whose diameter decreases as one approaches the injection end (the tip).

The surgeon presses the piston whose end pushes the implant; the latter is forced more and more into the tip of the injector and ends up coming out of the injector completely folded. Thus, it is possible to inject an implant with a diameter of more than 6 mm through an incision with a size smaller than 3 mm. Very high stresses are then exerted on the implant during the transfer. To limit the injection force and enable the implant to come out of the tip without damage, it is necessary to optimise the geometry of the tip and the nature of the support, on the one hand, and to use a “lubricant”, on the other hand. In the following description, the terms “intraocular implant injection device”, “injection system” or “ophthalmic injector” will be used interchangeably. For example, the patent applications EP1173115, EP2344073 or WO/2007 054644 and WO/2007 021412 describe injection systems with different geometric designs. Many commercially-available injectors feature the different shapes and/or mechanical inventions described in the patents. Mention may be made, for example, of one-piece injectors (connected tips and injection bodies) of the Skyjet® type commercialised by Carl Zeiss Meditec or the Accujet®, Navijet® or Viscojet® injectors from Medicel AG where the tip is removable and consists of an injection chamber in which the implant is placed, which is immediately folded and passes through a cannula to be injected, or the Monarch® type injectors from Alcon laboratories where the tip is removable and the implant is slipped into the tip without prior folding.

The mechanical characteristics of the constituent material of the tip also have an influence on injectability. Indeed, the thermoplastic material used for the manufacture of the tip should allow for some deformability to support the stresses imparted on the implant while presenting a good rigidity. In addition, the used thermoplastic materials should be able to be injected at high rates. Preferably, these materials are selected from among the family of polypropylenes, polyamides, polyurethanes or polyesters and more particularly from the family of polypropylenes and polyamides.

However, the selection of the material of the tip and/or the cartridge as well as the optimisation of its geometry are not enough to inject implants via micro-incisions in a satisfactory manner. It is imperative to use a lubricant enabling the implant to slip in the tip and/or the cartridge. Alternatively, the tip and the cartridge may be connected and form one single element. This alternative is covered by the expression “the tip and/or the cartridge” or “tip/cartridge”. Two different approaches enabling slipping are described in the literature. The first one relates to the use of a migration agent (“Blooming agent”) integrated by compounding into the thermoplastic material. It consists of a surfactant organic molecule with a low molar mass of the glycerol monostearate (GMS) type which is compounded with polypropylene or polyamide. It is evenly distributed in the thermoplastic support just after injection and ends up migrating to the surface of the support after several days, even several weeks. This migration phenomenon is related to the small size of the surfactant molecule which is mobile compared to the macromolecular chains. For example, the patent U.S. Pat. No. 6,733,507 describes polypropylene cartridges containing a lubricating agent which migrates to the surface by a phenomenon of migration (“blooming”). This approach has two major drawbacks.

The first one is the presence of white traces on the injected implants. They are due to the migration agent which is not bound to the surface of the tip and which is carried away during the injection. Indeed, the used migration agents are not water-soluble and their elimination is possible only after numerous rinses once the implant has been injected.

The second drawback is related to the phenomenon of migration of the migration agent to the surface of the support. This migration could last several days, even several weeks, depending on the conditions of implementation (injection of the part), the storage temperature, the post-treatment, the sterilisation conditions, etc. . . . before a sufficient amount of lubricant cumulates at the surface of the injection cartridge. Hence, the quality of the lubrication will depend on the waiting time between the manufacture of the injector and the use thereof by the practitioner. If this period is too short, the lubrication is not ensured satisfactorily and if it is too long, the implant is covered with white spots (presence of the migration agent) during the injection. To overcome this drawback, the application WO2005/018505 suggests heat-treating the parts in order to accelerate the migration phenomenon and to obtain a quasi-stable state (sufficient amount of lubricant at the surface of the support) allowing repeatable injections. Another way described by the U.S. Pat. No. 7,348,038 suggests a plasma treatment to ensure a physical bond of the migration agent to the cartridge.

The non-repeatability of lubrication and the presence of whitish spots on the implants during injection represent the main drawbacks of the use of migration agents.

The second approach enabling slipping of the implants in the tips/cartridges of the injectors is the set-up of a hydrophilic coating inside the tip. The principle of lubrication consists in swelling the hydrophilic coating by the addition of a viscous product (hyaluronate or hydroxypropylmethyl cellulose solution) and thus sliding over a film of water formed at the interface.

The patent applications or patents JP5690838, JP3254752, U.S. Pat. No. 5,716,364, EP1949871, WO96/22062, WO2007/030009, WO2010/118080, U.S. Pat. Nos. 7,687,097, 7,348,038, WO2010/059655, for example, describe the possibility of making a coating allowing reducing or eliminating the friction between the implant and the cartridge.

The hydrophilic coating is bonded either by covalent chemical bonds or by physical bonds to the surface of the tip and/or cartridge.

As regards coatings bonded by physical interactions to the support, an activation of the surface by a plasma or corona or other photochemical methods, is systematically recommended as disclosed, for example, in the patent applications or patents JP5690838, JP3254752, EP1949871, U.S. Pat. No. 5,716,364, WO2010/059655, WO2010/118080 or U.S. Pat. No. 7,348,038. Once the surface is activated, the polymer in solution is deposited and the solvent is evaporated afterwards. The hydrophilic polymers are selected from among polyacrylic acid, polymethacrylic acid, polyvinylacetate, polyacrylamide, polyvinylpyrolidone and their copolymer, and sometimes a mixture of several ones of these polymers. However, it happens that the constituent polymers of the coating peel off the support or are solubilised by the viscous product and are carried away by the implant into the eye. If the practitioner does not flush the eye satisfactorily after set-up of the implant, the residues of the coating may ultimately cause inflammation.

The application FR2986532 describes a metastable polymer composition comprising a mixture of at least one constituent polymer and at least one partially miscible or compatible functional copolymer, wherein the constituent polymer is a thermoplastic polymer with a high molar mass and having a glass transition temperature (Tg) or a melting point (Tf) higher than or equal to 80° C., present in the mixture in a mass proportion comprised between 85% and 99.5%, and the functional copolymer, partially miscible or compatible with the constituent polymer, has a lower molar mass and a glass transition temperature (Tg) or a melting point (Tf) higher than or equal to 40° C.

This mixture allows developing a material for the manufacture of a biomedical device having slippery properties at its surface without requiring coating of a material. The principle is based on the migration of the functional copolymer within the constituent polymer, which requires the use of a functional copolymer with a low molar mass, capable of migrating to the surface of the constituent polymer.

As indicated on p. 16, I.8-15, the aim sought in the application FR2986532 differs completely from the “coating” approach (p. 16, I.8-15): “The present invention is completely different from the “coating” approach (“coating”) The latter, which consists in depositing a polymer film at the surface of the device or of some of its elements, requires carrying out several operations including activation of the surface, the removal of the polymer in solution and the evaporation of the solvent. The approach suggested in the present invention allows avoiding all these steps in the manufacturing process and leads to a simplification of the method to the sole step of implementation by injection.”

Thus, the application FR2986532 does not relate to the development of a slippery coating for an intraocular implant injection device.

Moreover, coatings covalently bonded to the cartridge and/or to the tip are described, in particular in the patent applications or patents U.S. Pat. No. 7,687,097 and WO96/22062. The recommended approach consists in immersing the cartridge, pretreated or not by plasma, in a precursor having reactive functions, for example acrylate groups, then by a radical route (thermal or UV irradiation) initiating the polymerisation of the precursor, some chains of which are brought to react with the radicals formed at the surface of the cartridge.

Other patent applications or patents such as U.S. Pat. Nos. 6,238,799, 6,866,936 or WO96/23602 describe the covalent grafting of hydrophilic polymers, or hydrogels, onto supports for medical devices. The principle is based on the development of interpenetrating networks consisting of a polyurethane combined with another hydrophilic polymer.

However, the covalent grafting of the coating over the surface of the tip and/or cartridge of the ophthalmic injector is a long and expensive operation which limits the interest of the approach.

Hence, it is sought to develop a slippery coating which does not require a chemical grafting at the surface of the tip and/or the injector cartridge and which does not peel off during the injection of the implant.

### SUMMARY OF THE INVENTION

According to a first aspect, the invention relates to the use of a polymer able to form a slippery coating, of formula (I):

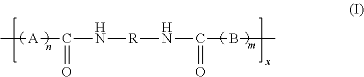

wherein:
A represents a —(CH₂—CH₂—O)— group
R is selected from among the following groups:

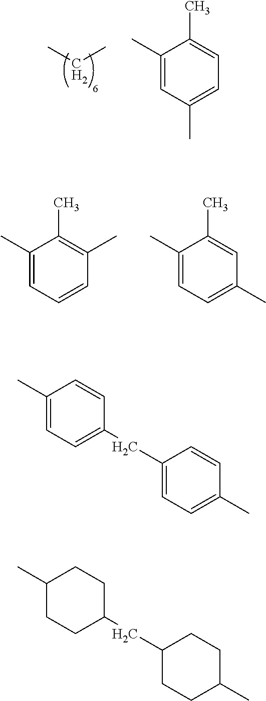

B represents an ether or ester group as defined hereinbelow:

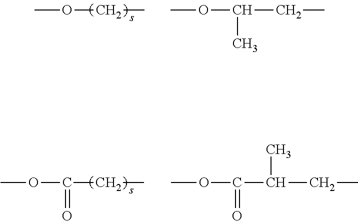

n is an integer comprised between 10 and 500, preferably between 20 and 300;
m is an integer comprised between 1 and 50, preferably between 1 and 20;
s is an integer comprised between 4 and 18, preferably between 4 and 10; and
x being an integer selected so that the number-average molar mass of the polymer is comprised between 20,000 g/mol and 1,000,000 g/mol, to coat at least one portion of an intraocular implant injection device.

In particular, the invention relates to the use of said polymer for coating the inner surface of one or more portion(s) of an intraocular implant injection device, allowing conferring a slippery character thereon, as well as the method for coating said surface, allowing for a good adhesion of the polymer.

The invention also relates, as a new product, to a polymer of formula (I)

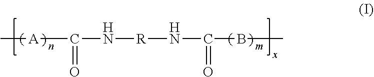

wherein
A, R and B are as defined hereinabove,
n, m and s are as defined hereinabove,
x is an integer selected so that the number-average molar mass of said polymer is greater than 100,000 g/mol and up to 1,000,000 g/mol, and said polymer has a melting point from 40° C. to 60° C.,
   as well as the use thereof for coating at least one portion of an intraocular implant injection device, in particular for coating the inner surface of one or more portion(s) of said injection device.

Another object of the invention is a portion of an intraocular implant injection device, whose inner surface is coated with a polymer as defined hereinabove, as well as an intraocular implant injection device comprising at least one portion whose inner surface is coated with said polymers.

### DETAILED DESCRIPTION

The present invention aims to provide slippery coatings for intraocular implant injection devices, which are hydrophilic enough to be swollen by viscous products and fill their role of lubricant while being hydrophobic enough to avoid any unintentional solubilisation and dispersion of said coating in the eye of the patient, thus avoiding post-operative problems.

The invention consists in developing a coating for an ophthalmic injector based on a polymer whose molar masses and hydrophilic-hydrophobic balance are perfectly adjusted in order to form a film at the surface of an ophthalmic injector tip; this film has the specificity of being slippery, on the one hand, and perfectly water-resistant, on the other hand. This water resistance avoids any entrainment of the polymer into the eye.

Thus, according to the invention, said polymer should have a molar mass that is high enough to prevent solubilisation thereof in water during use thereof. The “slippery” character of the coating according to the invention could be measured, for example, by using a one-piece ophthalmic injector entirely manufactured by injection/moulding from said composition or an injector consisting of two parts in which only the loading cartridge and the tip are manufactured from said composition. The thrust force on the piston of the injector necessary to eject the implant loaded into the cartridge of the injector is measured, through a tip whose outlet diameter is smaller than 3 mm, for example 2 mm. This measurement may be performed by compression by means of an Instron 3367 type dynamometer equipped with a 0.5 kN sensitivity force sensor at a speed of 4 mm/s and at room temperature. It is considered that the slippery property is significant for force values comprised between 5 and 10 N. It is moderate for force values higher than 10 N and lower than 15 N, and low for force values higher than 15 N.

Thus, it is considered that the deposition of a coating according to the invention confers a “slippery” character on an ophthalmic injector when a practitioner injects without damage an implant with a diopter less than or equal to 30 D into the eye of a patient, by applying a force lower than or equal to 20N.

The invention also relates to the method for implementing the coating. The method according to the invention enables a good adhesion of said polymer to the surface of the tip and/or of the cartridge of the injector to avoid said coating being peeled off and carried away by the implant, during injection thereof, into the eye of the patient.

According to the invention, said coating is deposited over the surface of the tip and/or the cartridge of the injector. Attachment thereof is essentially ensured by the hydrophobic blocks of said polymer which have a very good chemical compatibility with the constituent material of the tip and/or the cartridge of the injector. The hydrophilic blocks of said polymer, which have little or no interactions with the surface of the tip and/or the cartridge of the injector, allow obtaining the desired slippery properties.

One of the objectives of this invention is to develop a polymer having hydrophobic blocks having strong enough interactions with the surface of the tip and/or the cartridge of the injector (without there being any interpenetration between the polymer chains) and hydrophilic blocks to obtain sufficient slippery properties for the injection of intraocular implants.

Advantageously, the slippery coating according to the invention has the following properties:
a hydrophilic-hydrophobic balance and adjusted molar masses,
film-forming properties,
good affinity with the support made of polypropylene or polyamide.

According to a first aspect of the invention, the constituent polymer of the coating according to the invention may be selected from among the family of polyurethanes. The adjustment of the hydrophilic-hydrophobic balance of this polymer type may be obtained by the selection of the nature and the ratio of the selected polyols and di-isocyanates.

Preferably, the polymer according to the invention comprises a predominant hydrophilic part, preferably at least 70% by mass of hydrophilic monomer units, in particular between 80 and 95% by mass of hydrophilic monomer units. Polyurethanes also have good film-forming properties; besides, they are used as varnish for many applications. Finally, their polar nature and their ability to create hydrogen bonds enable them to have high affinity with many supports. Hence, according to a first aspect, the invention relates to the use of a polymer, of formula (I):

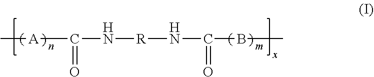

wherein:
A represents a —(CH₂—CH₂—O)— group
R is selected from among the following groups:

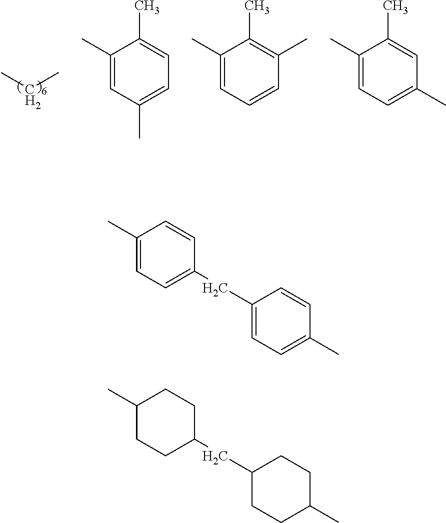

B represents an ether or ester group as defined hereinbelow:

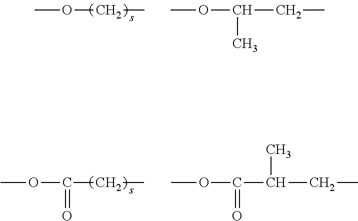

n is an integer comprised between 10 and 500, preferably between 20 and 300;
m is an integer comprised between 1 and 50, preferably between 1 and 20;
s is an integer comprised between 4 and 18, preferably between 4 and 10; and
x being an integer selected so that the number-average molar mass of the polymer is comprised between 20,000 g/mol and 1,000,000 g/mol, to form a slippery coating over at least one portion of an intraocular implant injection device.

Advantageously, the inner surface of at least one portion of an intraocular implant injection device, in particular the tip and/or the cartridge, is coated with said polymer.

According to a preferred aspect, x is an integer selected so that the number-average molar mass of said polymer is comprised between 50,000 g/mol and 1,000,000 g/mol, preferably between 100,000 g/mol and 1,000,000 g/mol, in particular between 50,000 g/mol and 500,000 g/mol, in particular greater than 100,000 g/mol, and in particular between 110,000 g/mol and 500,000 g/mol. According to the invention, the polymer of formula (I) may be obtained with the adequate hydrophilic-hydrophobic balance by reacting a hydrophilic diol of the dihydroxy-telechelic polyethylene oxide type with a hydrophobic diol of the HO—B—OH type, B being as defined hereinabove, in the presence of a diisocyanate. For example, said diisocyanate may be selected from among the following compounds:

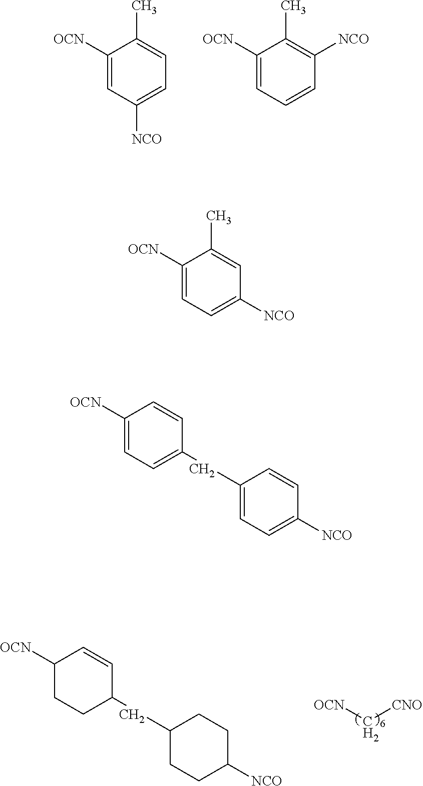

An important parameter allowing obtaining an adequate hydrophilic-hydrophobic balance is the mass ratio between the dihydroxy-telechelic polyethylene oxide acid and the hydrophobic diol. Preferably, this ratio is comprised between 99/1 and 70/30, and more particularly between 97/3 and 80/20.

According to an advantageous aspect, said polymer has a melting point from to 60° C., preferably about 50° C.

The invention also relates, as a new product, to a polymer able to form a slippery coating, of formula (I)

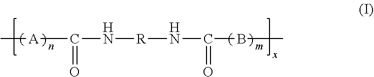

wherein
A represents a —(CH₂—CH₂—O)— group
R is selected from among the following groups:

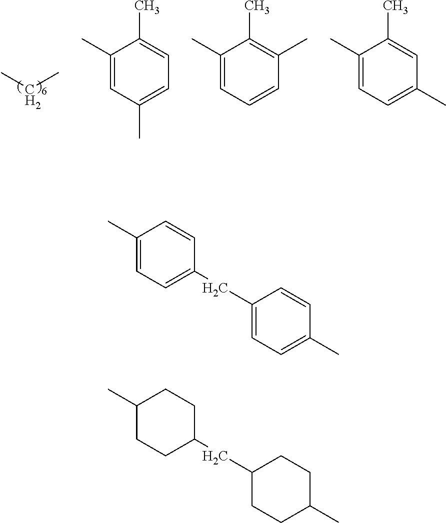

B represents an ether or ester group as defined hereinbelow:

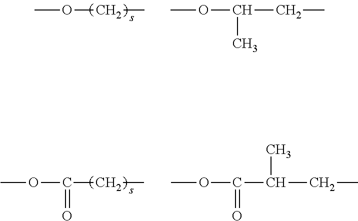

n is an integer comprised between 10 and 500, preferably between 20 and 300;
m is an integer comprised between 1 and 50, preferably between 1 and
s is an integer comprised between 4 and 18, preferably between 4 and 10;
x is an integer selected so that the number-average molar mass of said polymer is greater than 100,000 g/mol and up to 1,000,000 g/mol, in particular greater than 100,000 g/mol and up to 500,000 g/mol, in particular between 110,000 and 500,000 g/mol, and said polymer has a melting point from 40° C. to 60° C., preferably about 50° C.

According to the invention, an advantageous aspect of the constituent polymers of the coating, as described hereinabove, is their ability to form a film. Indeed, the slippery character of the coating is also related to the formation of a smooth and homogeneous film. It has been found that polymers with a number-average molar mass greater than 20,000 g/mol, in particular greater than 50,000 g/mol, in particular greater than 100,000 g/mol, lead to more homogeneous films. The selection of the molar mass of the implemented polymers also conditions the resistance to dissolution in water. The higher the molar mass, the greater the resistance to dissolution in water will be. Preferably, the implemented polymers according to the invention have number-average molar masses greater than g/mol and lower than 1,000,000 g/mol, and in particular comprised between 50,000 g/mol and 500,000 g/mol, and still more preferably greater than 100,000 g/mol.

According to a subsequent aspect of the invention, the method for depositing the polymer film over the constituent material of the tip and/or cartridge of the ophthalmic injector is important to avoid peeling of the coating and it being entrained into the eye of the patient. Preferably, to ensure good adhesion of the coating to the tip and/or the cartridge of the ophthalmic injector, it is proceeded with a specific treatment of the latter. In a first step, all traces of deposits, such as grease or particles, at the surface of the tip and/or the cartridge are removed by washing, for example by washing with alcohol followed by rinsing with pure water. Afterwards, the surface of the material is subjected to a treatment enabling a strong oxidation, in order to increase the surface tension of the support. Thus, the tip and/or the cartridge is (are) treated with corona, oxygen plasma or air. The presence of functions created thanks to oxidation at the surface of the tip and/or the cartridge enables the easy creation of hydrogen-type bonds with the coating. Indeed, the systematic presence of urethane bonds in the coating (assisted by ester bonds for the coatings that contain them) ensures very good adhesion to the support treated with corona, or plasma with argon, oxygen or air, in particular thanks to hydrogen bonds.

The coating implementing the polymers of formula (I) according to the invention, as described hereinabove, is preferably carried out by the solvent route. The polymer is solubilised at a concentration ranging from 0.5% to 3% by mass in a solvent of the polymer, and preferably in a mixture consisting of water and ethanol. The tip and/or cartridge is (are) filled with this solution. After a few seconds, for example about 3 seconds, the excess is evacuated; the polymer deposited at the surface of the tip and/or cartridge is then dried. The film thus deposited is smooth and homogeneous.

The injection tests carried out with these coated tips/cartridges according to the different aspects of the invention have enabled an easy injection of the implants, including through small diameters (<3 mm) without peeling or solubilisation of the coating. No trace of the polymer forming the coating has been found by HPLC analysis neither on the surface of the implants nor in the medium in which the implants have been injected.

The above-described general and particular aspects of the invention apply indifferently to the polymers of formula (I), to the use thereof to form a slippery coating over at least one portion of an intraocular implant injection device and to the method for obtaining them, as well as to the method for coating the inner surface of at least one portion of an intraocular implant injection device implementing them and to the device comprising at least one portion whose inner surface is coated with said polymer.

### EXAMPLES

### Example 1: Preparation of a Polyurethane Whose Diol Part is Composed of Polyethylene Oxide and Poly(ε-Caprolactone)

400 g of a dihydroxy-telechelic POE having a number-average molar mass of 6,000 g/mol (6.67×10⁻² mol) and 100 g of a dihydroxy-telechelic poly(ε-caprolactone) (8×10⁻² mol) with a molar mass=1,250 g/mol are dried under vacuum for 2 hours at 100° C., then solubilised in 1.45 litres of butanone dried beforehand over CaCl₂). The solution is heated up to 85° C. at the time of introduction of 38.43 g of 4,4′-methylene biscyclohexyl di-isocyanate (14.67×10⁻² mol). 0.5 g of a Bismuth-based catalyst is added 5 minutes after the end of the isocyanate introduction.

The reaction is terminated after 19 hours by the addition of 1 ml of ethanol. The solution is cooled down to room temperature then diluted in 1.5 l of acetone then precipitated in 8 l of heptane.

The number-average molar mass of the obtained polymer (PS equivalent) is 250,000 g/mol.

### Example 2: Preparation of a Polyurethane Whose Diol Part is Composed of Polyethylene Oxide and Decanediol

400 g of a dihydroxy-telechelic POE having a number-average molar mass of 6,000 g/mol (6.67×10⁻² mol) and 10 g of decanediol (5.74×10⁻² mol) with a molar mass=174 g/mol are dried under vacuum for 2 hours at 100° C., then solubilised in 1.45 litres of 2-butanone dried beforehand over CaCl₂). The solution is heated up to 85° C. at the time of introduction of 20 g of toluene di-isocyanate (11.5×10⁻² mol). 0.5 g of a Bismuth-based catalyst is added 5 minutes after the end of the isocyanate introduction.

The reaction is terminated after 19 hours by the addition of 1 ml of ethanol. The solution is cooled down to room temperature then diluted in 1.5 l of acetone then precipitated in 8 l of heptane.

The number-average molar mass of the obtained polymer (PS equivalent) is 85,000 g/mol.

### Example 3: Preparation of a Polyurethane Whose Diol Part is Composed of Polyethylene Oxide and Propylene Polyoxide

400 g of a dihydroxy-telechelic POE having a number-average molar mass of 6,000 g/mol (6.67×10⁻² mol) and 100 g of dihydroxy-telechelic propylene polyoxide (10×10⁻² mol) with a molar mass=1,000 g/mol are dried under vacuum for 2 hours at 100° C., then solubilised in 1.45 litres of 2-butanone dried beforehand over CaCl₂). The solution is heated up to 85° C. at the time of introduction of 37.05 g of toluene di-isocyanate (16.67×10⁻² mol). 0.5 g of a Bismuth-based catalyst is added 5 minutes after the end of the isocyanate introduction.

The reaction is terminated after 19 hours by the addition of 1 ml of ethanol. The solution is cooled down to room temperature then diluted in 1.5 L of acetone then precipitated in 8 L of heptane.

The number-average molar mass of the obtained polymer (PS equivalent) is 63,000 g/mol.

### Example 4: Treatment of the Tips and Cartridges Before Application of the Coating According to the Invention

The tips/cartridges are washed twice with ethanol then rinsed twice with pure water. Afterwards, they are dried with compressed air.

The device used for the plasma is a PlasmaNet MWGO. The used gas is air at a pressure of 0.4 mbar. The gas use time is 20 s, and the plasma treatment lasts 195 s at a power of 3,000 W.

The surface tension of the tips/cartridges is determined using test inks after the plasma treatment to ensure effectiveness thereof. Indeed, at the end of the cycle, samples of tips/cartridges are collected; ACOTEST 56 mN/m ink is deposited at the surface of the supports. The plasma treatment is validated if the ink spreads perfectly.

### Example 5: Application of the Coating According to the Invention

The polymer of Example 1 is solubilised at 1% in a 70% water 30% alcohol mixture for 24 hours at room temperature. Afterwards, the solution is filtered through a 0.2 μm filter. The solution is ready for application.

The tips/cartridges are filled with the solution prepared hereinabove. The excess is evacuated under vacuum after about 3 seconds of contact.

Afterwards, the deposit is dried at 40° C. for 24 h. The formed film is smooth and homogeneous.

### Example 6

The injection tests carried out with the coatings made from different polymers are reported in Table 1 hereinbelow.

The measurement has been performed by compression by means of an Instron 3367 type dynamometer equipped with a force sensor with a 0.5 kN sensitivity at a speed of 4 mm/s and at room temperature.

TABLE 1

| | Lens | | Nature | |
|---|---|---|---|---|
| | Nature/ | Tip | of the | Force IOL |
| Reference | Diopter | diameter | coating | outlet (N) |
| DIS15042B | Hydrophilic/24.5 | 2.6 mm | Example 2 | 12 |
| | | | | 14 |
| | | | Average | 13 |
| | | | Standard- | 1 |
| | | | deviation | |
| DIS15042A | Hydrophilic/24.5 | 2.6 mm | Example 3 | 9 |
| | | | | 9 |
| | | | | 8 |
| | | | Average | 9 |
| | | | Standard- | 0 |
| | | | deviation | |
| DIS15047D3 | Hydrophilic/17 | 2.0 mm | Example 1 | 10 |
| | | | | 10 |
| | | | Average | 10 |
| | | | Standard- | 0 |
| | | | deviation | |
| DIS15047D1 | Hydrophilic/17 | 2.0 mm | Example 3 | 10 |
| | | | Average | 10 |
| | | | Standard- | 0 |
| | | | deviation | |
| DIS15055B1 | Hydrophilic/17 | 2.0 mm | Example 1 | 11 |
| DIS15047D4 | Hydrophilic/28 | 2.0 mm | Example 3 | 13 |
| | | | | 13 |
| | | | Average | 13 |
| | | | Standard- | 0 |
| | | | deviation | |
| DIS15055B2 | Hydrophilic/28 | 2.0 mm | Example 1 | 12 |
| | | | | 10 |
| | | | Average | 11 |
| | | | Standard- | 1 |
| | | | deviation | |

The results show that the deposited coating confers a slippery character to the injector allowing injecting an intraocular implant with at least 6 mm diameter through a tip with a 2 mm diameter.

### Example 7

50 hydrophilic implants with a 28 diopter have been injected using an injector having a tip with a 2 mm diameter (treated according to the protocol of Example in a pure water bath at 32° C.

The water bath containing the implants has been concentrated using a rotavator and then lyophilised. The residual has been analysed by HPLC (water/acetonitrile). No trace of the coating of Example 1 has been found in the analysed product.

1. A method for forming a slippery coating over at least one portion of an intraocular implant injection device, comprising the step of depositing a solution of a polymer able to form a slippery coating, of formula (I): 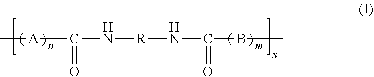
wherein:
A represents a —(CH₂—CH₂—O)— group;
R is selected from among the following groups; 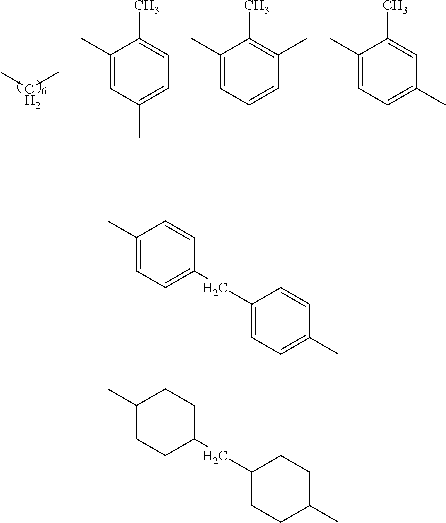
B represents an ether or ester group as defined hereinbelow: 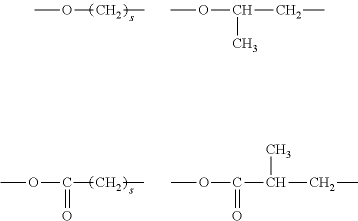
n is an integer comprised between 10 and 500;
m is an integer comprised between 1 and 50;
s is an integer comprised between 4 and 18; and
x being an integer selected so that the number-average molar mass of the polymer is comprised between 50,000 g/mol and 1,000,000 g/mol,
on the inner surface of one or more portion(s) of an intraocular implant injection device.

2. The method according to claim 1, wherein, in formula (I), x is an integer selected so that the number-average molar mass of said polymer is comprised between 100,000 g/mol and 1,000,000 g/mol.

3. The method according to claim 1, wherein said polymer of formula (I) is obtained by reacting a hydrophilic diol of the dihydroxy-telechelic polyethylene oxide type with a hydrophobic diol selected from among poly(ε-caprolactone) or decanediol in the presence of a diisocyanate.

4. The method according to claim 1, wherein said polymer of formula (I) is obtained by reacting a hydrophilic diol of the dihydroxy-telechelic polyethylene oxide type with a hydrophobic diol in the presence of a diisocyanate selected from among 4,4′-methylene biscyclohexyl diisocyanate and toluene diisocyanate.

5. (canceled)

6. The method according to claim 1, wherein said portion is the tip and/or the cartridge of an ophthalmic injector.

7. A method for obtaining a polymer of formula (I) 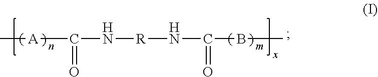
wherein:
A represents a —(CH₂—CH₂—O)— group;
R is selected from among the following groups: 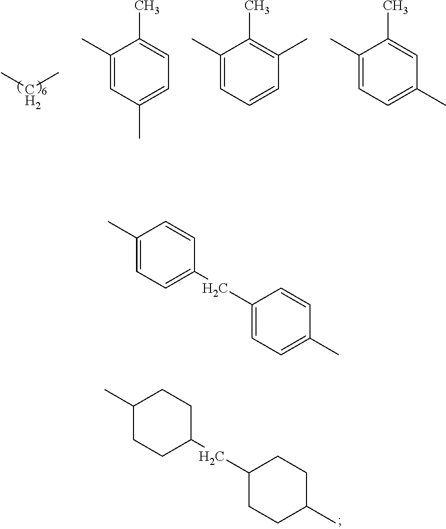
B represents an ether or ester group as defined hereinbelow: 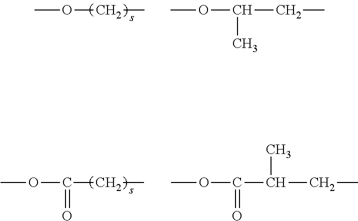
n is an integer comprised between 10 and 500:
m is an integer comprised between 1 and 50;
s is an integer comprised between 4 and 18; and
x being an integer selected so that the number-average molar mass of the polymer is comprised between 50,000 g/mol and 1,000,000 g/mol;
comprising reacting a hydrophilic diol of dihydroxy-telechelic polyethylene oxide type with a hydrophobic diol HO—B— OH wherein B is as defined in claim 1 in the presence of a diisocyanate, and wherein the mass ratio between the dihydroxy-telechelic polyethylene oxide and the hydrophobic diol is comprised between 99/1 and 70/30.

8. A method for coating the inner surface of one or more portion(s) of an intraocular implant injection device, comprising the following steps:
subjecting the inner surface of the portion(s) of the intraocular implant injection device to be coated, to a strong oxidative treatment, and
depositing a solution of a polymer of formula (I) 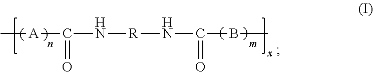
wherein:
A represents a —(CH₂—CH₂—O)— group;
R is selected from among the following groups: 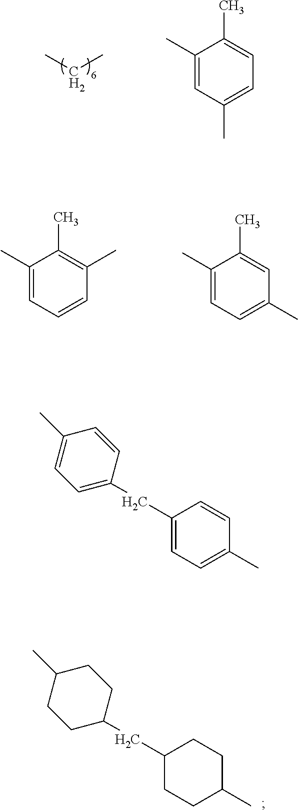
B represents an ether or ester group as defined hereinbelow: 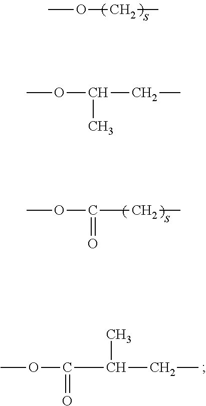
n is an integer comprised between 10 and 500;
m is an integer comprised between 1 and 50:
s is an integer comprised between 4 and 18; and
x being an integer selected so that the number-average molar mass of the polymer is comprised between 50,000 g/mol and 1,000,000 g/mol.

9. The method according to claim 8, wherein the strong oxidative treatment of the inner surface is a corona, oxygen plasma or air treatment.

10. The method according to claim 8, wherein the polymer of formula (I) is solubilised in a solvent of said polymer, at a concentration ranging from 0.5% to 3% by mass.

11. The method according to claim 8, wherein the portion of the injector to be coated is filled with the polymer solution and then dried, after evacuation of the excess.

12. The method according to claim 8, further comprising a prior step of specific treatment of the portion(s) of the intraocular implant injection device to be coated in order to eliminate all traces of deposits on the surface thereof.

13. A polymer able to form a slippery coating, of formula (I) 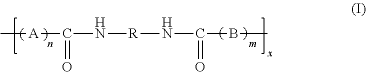
wherein
A represents a —(CH₂—CH₂—O)— group;
R is selected from among the following groups: 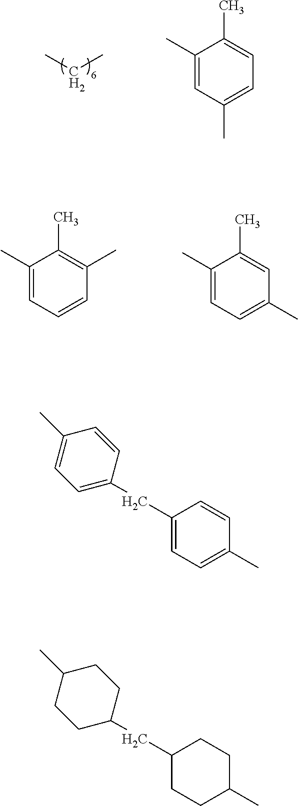
B represents an ether or ester group as defined hereinbelow 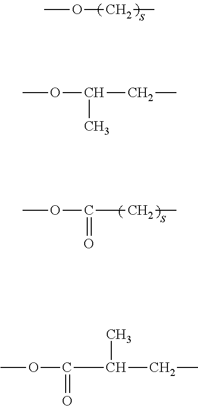
n is an integer comprised between 10 and 500;
m is an integer comprised between 1 and 50;
s is an integer comprised between 4 and 18;
x is an integer selected so that the number-average molar mass of said polymer is greater than 100,000 g/mol and up to 1,000,000 g/mol, and
said polymer has a melting point from 40° C. to 60° C.

14. (canceled)

15. (canceled)

16. (canceled)

17. A method for coating the inner surface of one or more portion(s) of an intraocular implant injection device, comprising the following steps:
subjecting the inner surface of the portion(s) of the intraocular implant injection device to be coated, to a strong oxidative treatment, and
depositing a solution of a polymer of formula (I) 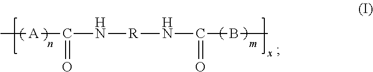
wherein
A represents a —(CH₂—CH₂—O)— group;
R is selected from among the following groups: 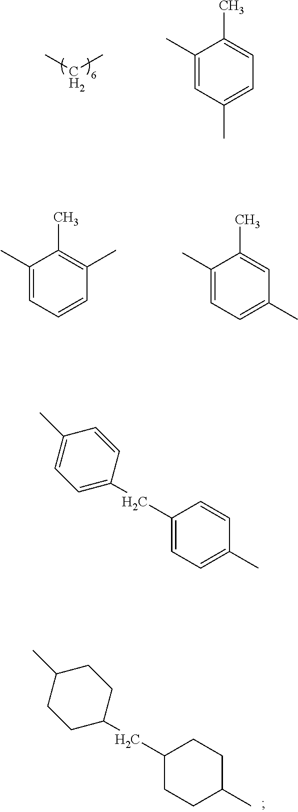
B represents an ether or ester group as defined hereinbelow: 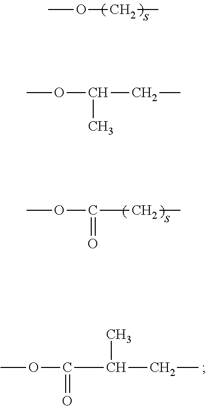
n is an integer comprised between 10 and 500:
m is an integer comprised between 1 and 50;
s is an integer comprised between 4 and 18;
x is an integer selected so that the number-average molar mass of said polymer is greater than 100,000 g/mol and up to 1,000,000 g/mol, and
said polymer has a melting point from 40° C. to 60° C.

18. The method according to claim 17, wherein the strong oxidative treatment of the inner surface is a corona, oxygen plasma or air treatment.

19. The method according to claim 17, wherein the polymer of formula (I) is solubilised in a solvent of said polymer, at a concentration ranging from 0.5% to 3% by mass.

20. A portion of an intraocular implant injection device, whose inner surface is coated with a polymer of formula (I) 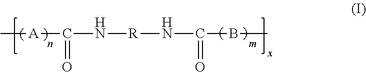
wherein:
A represents a —(CH₂—CH₂—O)— group;
R is selected from among the following groups: 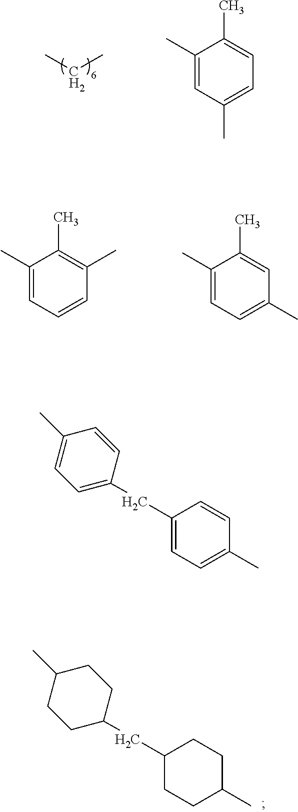
B represents an ether or ester group as defined hereinbelow 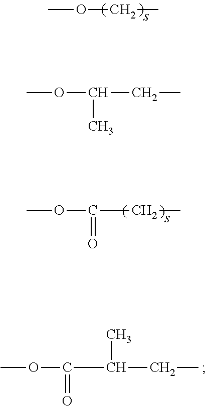
n is an integer comprised between 10 and 500;
m is an integer comprised between 1 and 50;
s is an integer comprised between 4 and 18; and
x being an integer selected so that the number-average molar mass of the polymer is comprised between 50,000 g/mol and 1,000,000 g/mol.

21. An intraocular implant injection device comprising at least one portion whose inner surface is coated with a polymer of formula (I) 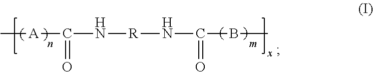
wherein:
A represents a —(CH₂—CH₂—O)— group;
R is selected from among the following groups: 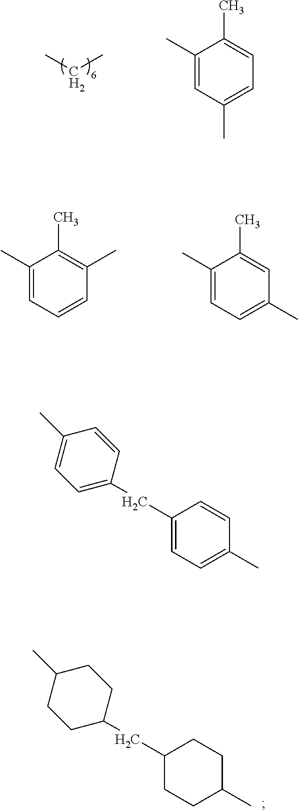
B represents an ether or ester group as defined hereinbelow: 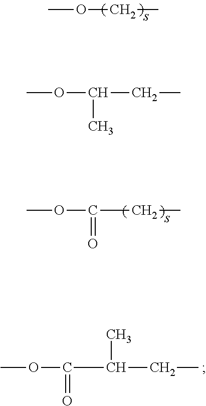
n is an integer comprised between 10 and 500;
m is an integer comprised between 1 and 50;
s is an integer comprised between 4 and 18; and
x being an integer selected so that the number-average molar mass of the polymer is comprised between 50,000 g/mol and 1,000,000 g/mol.

22. The method according to claim 1, wherein said polymer has a melting point from 40° C. to 60° C.

23. The method according to claim 7, wherein, in formula (I), x is an integer selected so that the number-average molar mass of said polymer is comprised between 50,000 g/mol and 500,000 g/mol.

24. The method according to claim 8, wherein, in formula (I), x is an integer selected so that the number-average molar mass of said polymer is comprised between 50,000 g/mol and 500,000 g/mol.

25. The method according to claim 8, wherein said polymer of formula (I) is obtained by reacting a hydrophilic diol of the dihydroxy-telechelic polyethylene oxide type with a hydrophobic diol selected from among poly(s-caprolactone) or decanediol in the presence of a diisocyanate.

26. A portion of an intraocular implant injection device according to claim 20, wherein, in formula (I), x is an integer selected so that the number-average molar mass of said polymer is comprised between 50,000 g/mol and 500,000 g/mol.

27. A portion of an intraocular implant injection device according to claim 20, wherein said polymer has a melting point from 40° C. to 60° C.

28. An intraocular implant injection device according to claim 21, wherein, in formula (I), x is an integer selected so that the number-average molar mass of said polymer is comprised between 50,000 g/mol and 500,000 g/mol.

29. An intraocular implant injection device according to claim 21, wherein said polymer has a melting point from 40° C. to 60° C.
